(19) United States
(12) Patent Application Publication
(10) Pub. No.: US 2022/0169608 A1
(43) Pub. Date: Jun. 2, 2022

(54) COMPOUND, ORGANIC SEMICONDUCTOR LASER AND METHOD FOR PRODUCING SAME

(71) Applicants: KYUSHU UNIVERSITY, NATIONAL UNIVERSITY CORPORATION, Fukuoka-shi, Fukuoka (JP); THE UNIVERSITY OF QUEENSLAND, St. Lucia ,Brisbane, Queensland (AU)

(72) Inventors: Sangarange Don Atula SANDANAYAKA, Fukuoka-shi, Fukuoka (JP); Adikari Mudiyanselage Chathuranganie SENEVIRATHNE, Fukuoka-shi, Fukuoka (JP); Toshinori MATSUSHIMA, Fukuoka-shi, Fukuoka (JP); Chihaya ADACHI, Fukuoka-shi, Fukuoka (JP); Ebinazar Benjamin NAMDAS, St Lucia, Brisbane, Queensland (AU); Shih-Chun LO, St Lucia, Brisbane, Queensland (AU); Van T. N. MAI, St Lucia, Brisbane, Queensland (AU); Atul SHUKLA, St Lucia, Brisbane, Queensland (AU); Ilene ALLISON, St Lucia, Brisbane, Queensland (AU); Sarah K. MCGREGOR, St Lucia, Brisbane, Queensland (AU)

(21) Appl. No.: 17/617,115

(22) PCT Filed: Jun. 5, 2020
(86) PCT No.: PCT/JP2020/022326

§ 371 Date: Dec. 7, 2021

(30) Foreign Application Priority Data
Jun. 7, 2019 (JP) 2019-107111

(51) Int. Cl. C07D 209/86; H01S 5/36; H01S 5/12

(52) U.S. Cl. CPC C07D 209/86; H01S 5/1221; H01S 5/36

(57) ABSTRACT

A compound of the formula (1) exhibits high photoluminescence quantum yields, high radiative decay constant and low ASE thresholds from solution-processed neat and blend films. Ar¹ and Ar² are aryl groups, L is a divalent group having a group of the formula (2), and R is H or a diarylamino group. At least one alkyl group having at least five carbon atoms which are bonded is present in the formula (1).

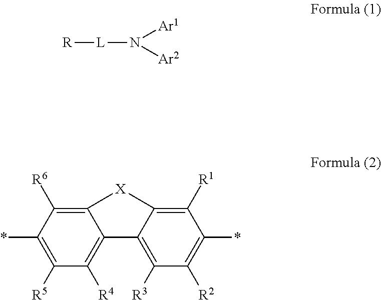

### TECHNICAL FIELD

The present invention relates to a compound, an organic semiconductor laser using it and a method for producing the organic semiconductor laser.

### BACKGROUND ART

Lasers incorporated with organic materials as gain media have a wide range of applications such as sensors, optical communications and spectroscopy. Compared to their inorganic counterparts, organic lasers offer many advantages such as low cost, light weight, high mechanical flexibility, ultrashort pulse and high wavelength-tunability. In addition, if the organic semiconductor materials are soluble in common organic solvents, these dyes can be processed using simple, fast, room-temperature manufacturing techniques such as spin-coating, dip-coating, ink-jet printing and blade-coating. Solution processability is highly desirable to progress toward low-cost and large-area organic lasers for commercial applications, especially in the area of disposable lasers.

Currently, all organic lasers are optically pumped using short excitation pulses with typical pulse width ranging from 100 fs to 10 ns. Optical losses due to absorptions of triplet excited-states at lasing wavelength has been consistently highlighted as a detrimental factor impeding long pulsed excitation in organic laser. Optical excitation, which is a spin-conserving process, initially generates only singlet excited-states. Any triplet excited-states present in optical excitation are generated indirectly via intersystem crossing. In short pulsed photoexcitation of organic fluorescent dyes, the population of triplet excited-states is insignificant and given time to dissipate (via non-radiative decays back to the ground state) prior to the arrival of next photoexcitation pulse. Therefore, in short pulse photoexcitation (fs to ns range), triplet-induced optical losses are negligible. However, in long pulse regime, such as in quasi-continuous wave (qCW) and continuous wave (CW) operation i.e. operation in ms range, the accumulation of triplet excited-state becomes more prominent due to its much longer lifetime (μs to ms) compared to the singlet excited-states' short lifetime (typically in ns). Consequently, the triplet excited-state population and its associated triplet-induced optical losses increase significantly as the pulse duration increases, making CW lasing in organic semiconductor film extremely challenging. Nonetheless, the aim of demonstrating organic lasers operating in qCW and CW modes has continuously attracted great interests in the field because its successful demonstration will significantly extend the scopes of potential applications, especially in applications requiring beam duration in second ranges, such as lighting or analytical applications and finally realisation of injection lasing in organic materials.

So far, significant efforts have been made toward the realisation of organic lasers operating in qCW and CW modes. This includes new device architectures using such as new DFB resonators to further decrease lasing thresholds and/or minimise irreversible photodegradation, the use of triplet quencher additives to remove triplet-induced losses, and novel material development with low amplified spontaneous emission (ASE) thresholds, low triplet yields under photoexcitation (with high PLQY), and/or minimising triplet absorption at lasing wavelengths. However, in these approaches, a commercial laser dyes (e.g., Rhodamine etc.) in an inert polymer matrix (such as PMMA) were employed and the sample were rotated (mimicking jet stream of laser dye solution in liquid dye lasers) to avoid issue related to triplet pile-up.

Recently, Sandanayaka et al. reported a vacuum-deposited organic semiconductor laser dye, BSBCz, operates under very long photoexcitation pulse of 30 ms (see NPL 1). The demonstration of this qCW operation was attributed to the superior properties of BSBCz, including high PLQY (reaching 100% in CBP blend films), high radiative decay constant (1.0×10⁹ s⁻¹), and exceptionally low ASE threshold (0.3 μJ cm⁻²). However, comparable performances with the above-mentioned properties of BSBCz as well as CW operation in tens of ms range have not yet been demonstrated in solution-processable organic semiconductors.

### CITATION LIST

### Non Patent Literature

[NPL 1]
A. S. D. Sandanayaka, T. Matsushima, F. Bencheikh, K. Yoshida, M. Inoue, T. Fujihara, K. Goushi, J.-C. Ribierre, C. Adachi, Sci. Adv. 2017, 3, 1602570-1602578.

### SUMMARY OF INVENTION

This invention provides novel solution-processable organic semiconductor dyes to show CW lasing under long pulse photoexcitation (up to 10 ms). The inventors found that a new family of fluorene-based semiconductor dyes (e.g. SFCz, BSFCz and BSTFCz) exhibit excellent solubility in common organic solvents, high thermal stability, high photoluminescence quantum yields (PLQYs), high radiative decay constant (kᵣ up to 1.24×10⁹ s⁻¹), and extremely low solid-state ASE thresholds (Eₜₕ, ranging from 0.7 to 2.1 ρJ cm⁻²) from solution-processed blend films in a common organic light-emitting diode (OLED) host, tris(4-carbazoyl-9-ylphenyl)amine (TCTA). All compounds also show remarkably low neat-film ASE thresholds of 2.5-5.5 ρJ cm⁻². Distributed feedback (DFB) lasers based on the three new compounds were fabricated and characterised, using both second-order and mixed-order grating structures. The demonstration of lasing was confirmed by polarisation, as well as near-field and far-field interference effects. Transient absorption spectroscopy (TAS) was conducted to reveal extremely low excited-state absorptions at lasing wavelengths in BSFCz, which prompted our further investigation of the materials for CW operation. Impressively, solution-processed DFB grating lasers based on BSFCz demonstrated efficient lasing under long photoexcitation pulse up to 10 ms. These results indicate the great potential of the new series of materials as extremely low ASE thresholds and solution-processable organic semiconductor dyes. Most importantly, our results highlight the class of materials as a high-performing laser dye possessing many advantageous features that are not shared by existing organic semiconducting dyes, including solution-processability, low solid-state ASE threshold (0.7 ρJ cm⁻²), separation of triplet absorption from their lasing region, and demonstration of laser operation in a CW mode with excellent stability.

Specifically, this invention includes the followings:

[1] A compound represented by the following formula (1):

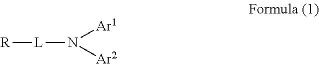

wherein:

Ar¹ and Ar² each independently represent a substituted or unsubstituted aryl group, and Ar¹ and Ar² may be chemically bonded to each other;

L represents a divalent group having at least three phenyl rings wherein the divalent group consists of at least one group represented by the formula (2) below, optionally at least one group represented by the formula (3) below, and optionally at least one group represented by the formula (4) below:

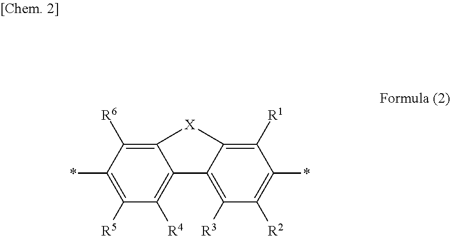

wherein X represents >C(R⁷)(R⁸), —O—, —S— or >N(R⁹); R¹ to R⁹ each independently represent a hydrogen atom or a substituent, R² and R³, and R⁴ and R⁵ may be taken together to form a ring, and each * represents a bonding site,

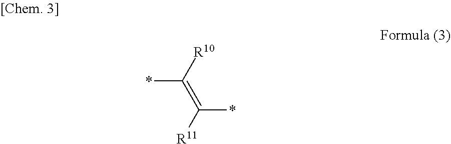

wherein R¹⁰ and R¹¹ each independently represent a hydrogen atom or a substituent,

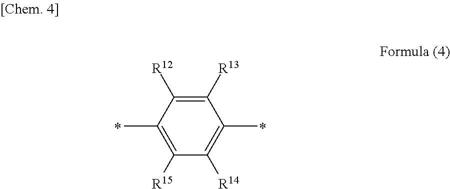

wherein R¹² to R¹⁵ each independently represent a hydrogen atom or a substituent, and R¹² and R¹³, and R¹⁴ and R¹⁵ may be taken together to form a ring,

R represents a hydrogen atom or a group represented by the following formula (5):

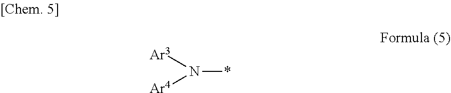

wherein Ar³ and Ar⁴ each independently represent a substituted or unsubstituted aryl group, and Ar³ and Ar⁴ may be chemically bonded to each other; and

wherein at least one solubilising alkyl or alkoxy or alkyl phenyl or alkoxy phenyl group having at least five carbon atoms which are bonded is represent in the formula (1).

[2] The compound according to [1], wherein L is a divalent group consisting of at least one group represented by the formula (2), at least one group represented by the formula (3), and at least one group represented by the formula (4).
[3] The compound according to [1] or [2], wherein L is a divalent group having a unit in which a group represented by the formula (2) and a group represented by the formula (3) are bonded.
[4] The compound according to any one of [1] to [3], wherein L is a divalent group having a unit in which a group represented by the formula (3) and a group represented by the formula (4) are bonded.
[5] The compound according to any one of [1] to [4], wherein L is a divalent group having a unit in which a group represented by the formula (2), a group represented by the formula (3) and at least one group represented by the formula (4) are bonded in this order.
[6] The compound according to any one of [1] to [5], having at least two alkyl or alkoxy groups having at least five carbon atoms which are bonded.
[7] The compound according to any one of [1] to [6], wherein L is a divalent group having at least one alkyl group having at least five carbon atoms which are bonded.
[8] The compound according to any one of [1] to [7], wherein X is >C(R⁷)(R⁸) or >N(R⁹) and R⁷ to R⁹ each independently represent an alkyl group having at least five carbon atoms which are bonded.
[9] The compound according to any one of [1] to [8], having two or more groups represented by the formula (2).
[10] The compound according to any one of [1] to [9], wherein —N(Ar¹)(Ar²) is a substituted or unsubstituted 9-carbazolyl group.
[11] The compound according to any one of [1] to [10], wherein R is a substituted or unsubstituted 9-carbazolyl group.
[12] The compound according to any one of [1] to [11], having a symmetrical structure.
[13] The compound according to any one of [1] to [12], having a structure represented by the following formula (6):

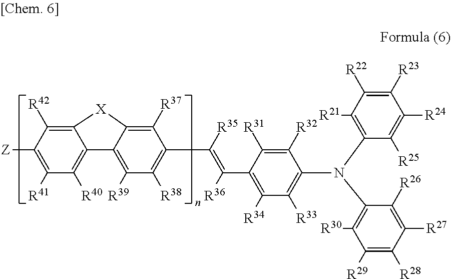

wherein X represents >C(R⁷)(R⁸), —O—, —S— or >N(R⁹), R⁷ to R⁹, R²¹ to R⁴² and Z each independently represent a hydrogen atom or a substituent, R²¹ and R²², R²² and R²³, R²³ and R²⁴, R²⁴ and R²⁵, R²⁵ and R²⁶, R²⁶ and R²⁷, R²⁷ and R²⁸, R²⁸ and R²⁹, R²⁹ and R³⁰, R³¹ and R³², R³³ and R³⁴, R³⁸ and R³⁹, and R⁴⁰ and R⁴¹ may be taken together to form a ring, and n is an integer of 1 to 12.
[14] The compound according to [13], wherein Z is represented by the following formula (7):

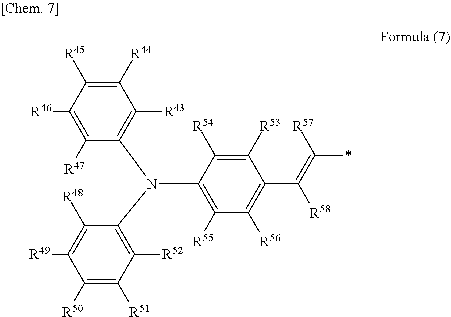

wherein R⁴³ to R⁵⁸ each independently represent a hydrogen atom or a substituent, R⁴³ and R⁴⁴, R⁴⁴ and R⁴⁵, R⁴⁵ and R⁴⁶, R⁴⁶ and R⁴⁷, R⁴⁷ and R⁴⁸, R⁴⁸ and R⁴⁹, R⁴⁹ and R⁵⁰, R⁵⁰ and R⁵¹, R⁵¹ and R⁵², R⁵³ and R⁵⁴, and R⁵⁵ and R⁵⁶ may be taken together to form a ring, and * represents a bonding site.
[15] Use of the compound of any one of [1] to [14] as an emitter in an organic semiconductor laser.
[16] An organic semiconductor laser comprising the compound of any one of [1] to [14] as an emitter.
[17] The organic semiconductor laser according to [16], having an optical resonator structure composed of a second-order Bragg scattering region.
[18] The organic semiconductor laser according to [16], having an optical resonator structure composed of a mixed-order Bragg scattering region.
[19] A method for producing an organic semiconductor laser comprising forming a layer having the compound of any one of [1] to [14] by a solution process.

### BRIEF DESCRIPTION OF DRAWINGS

FIGS. 1A-1C

Normalised UV-Vis absorption and photoluminescence spectra of solution (in toluene, dotted line), blend film (dash line, in 6 wt % TCTA) and neat film (solid line) of: (a) SFCz, (b) BSFCz, and (c) BSTFCz. Excitation wavelength=350 nm and 325 nm for solution and solid state, respectively.

FIG. 2(a)

ASE threshold achieved in 6 wt % blend film of SFCz. ASE threshold was estimated from the abrupt change in the slope of input-output intensity (in logarithmic-logarithmic scale) together with significant decrease in FWHM (left); photoluminescence spectra at excitation powers bellow and above ASE threshold showing spectral narrowing with increasing pump intensities (right).

FIG. 2(b)

ASE threshold achieved in 6 wt % blend film of BSFCz. ASE threshold was estimated from the abrupt change in the slope of input-output intensity (in logarithmic-logarithmic scale) together with significant decrease in FWHM (left); photoluminescence spectra at excitation powers bellow and above ASE threshold showing spectral narrowing with increasing pump intensities (right).

FIG. 2(c)

ASE threshold achieved in 6 wt % blend film of BSTFCz. ASE threshold was estimated from the abrupt change in the slope of input-output intensity (in logarithmic-logarithmic scale) together with significant decrease in FWHM (left); photoluminescence spectra at excitation powers bellow and above ASE threshold showing spectral narrowing with increasing pump intensities (right).

FIG. 3

ASE spectra overlapped with singlet excited-state absorption, and triplet excited-state absorption (magnified by 3 times) of BSFCz in toluene solution, showing essentially no excited-state absorptions at ASE wavelength.

FIGS. 4A-4C

SEM images of the fabricated mixed-order DFB gratings for each molecule and their grating periods; a) SFCz: grating period=256±5 and 128±5 nm; grating depth=65±5 nm; b) BSFCz: grating period=276±5 and 138±5 nm; grating depth=65±5 nm; c) BSTFCz: grating period=272±5 and 136±5 nm; grating depth=65±5 nm.

FIG. 5

The output intensity and emission spectra of BSFCz blend films with mixed-order grating structure as a function of pump intensity.

FIG. 6

The output intensity and emission spectra of BSFCz blend films with second-order grating structure as a function of pump intensity.

FIGS. 7A-7B

a) CW operational stability for BSFCz blend films of second-order DFB (excited power 668 W cm⁻²), and b) Output intensity and emission spectra with time interval.

FIGS. 8A-8B

a) CW operational stability for BSFCz blend films of mix-order DFB (excited power 668 W cm⁻²), and b) Output intensity and emission spectra with time interval.

### DETAILED DESCRIPTION OF INVENTION

The contents of the invention will be described in detail below. The elements of the invention may be described below with reference to representative embodiments and specific examples of the invention, but the invention is not limited to the embodiments and the examples. In the description, a numerical range expressed with reference to an upper limit and/or a lower limit means a range that includes the upper limit and/or the lower limit. The room temperature means 25° C.

The hydrogen atoms that are present in the compounds used in the invention are not particularly limited in isotope species, and for example, all the hydrogen atoms in the molecule may be ¹H, and all or a part of them may be ²H (deuterium (D)).

The alkyl group referred in the present application may be linear, branched or cyclic, and a linear or branched alkyl group is preferred. The alkyl group preferably has from 1 to 20 carbon atoms, more preferably from 1 to 12 carbon atoms, further preferably from 1 to 8 carbon atoms (e.g., a methyl group, an ethyl group, an n-propyl group, an isopropyl group, an n-butyl group, an isobutyl group, a tert-butyl group, an n-pentyl group, an isopentyl group, an n-hexyl group, an isohexyl group, an n-heptyl group, an n-octyl group, an n-nonyl group, an n-decyl group, an n-undecyl group and an n-dodecyl group; 2-ethylhexyl). Examples of the cyclic alkyl group include a cyclopropyl group, a cyclobutyl group, a cyclopentyl group, a cyclohexyl group, a cycloheptyl group, a bicylo[2.1.1]hexyl group and a bicyclo[2.2.1]heptyl group. The alkyl group may be substituted. Examples of the substituent in this case include an alkoxy group, an aryl group, an aryloxy group, an acyl group, an alkenyl group, a hydroxyl group, a halogen atom, a diarylamino group (including a 9-carbazolyl group), a cyano group, and a combination thereof, and preferred are an alkoxy group, an aryl group and an aryloxy group.

The alkoxy group referred in the present application may be linear, branched or cyclic, and a linear or branched alkoxy group is preferred. The alkoxy group preferably has from 1 to 20 carbon atoms, more preferably from 1 to 12 carbon atoms, further preferably from 1 to 8 carbon atoms (e.g., a methyloxy group, an ethyloxy group, an n-propyloxy group, an isopropyloxy group, an n-butyloxy group, an isobutyloxy group, a tert-butyloxy group, an n-pentyloxy group, an isopentyloxy group, an n-hexylyloxy group, an isohexyloxy group, an n-heptyloxy group, an n-octyloxy group, an n-nonyloxy group, an n-decyloxy group, an n-undecyloxy group, an n-dodecyloxy group, a 2-ethylhexyloxy group and a glycol group). Examples of the cyclic alkyloxy group include a cyclopropyloxy group, a cyclobutyloxy group, a cyclopentylox group, a cyclohexyloxy group, a cycloheptyloxy group, a bicylo [2.1.1]hexyloxy group and a bicyclo[2.2.1]heptyloxy group. The alkyloxy group may be substituted. Examples of the substituent in this case include an acytyl group, an aryl group, an aryloxy group, an acyl group, an alkenyl group, a hydroxyl group, a halogen atom, a diarylamino group (including a 9-carbazolyl group), a cyano group, and a combination thereof, and preferred are an alkyloxy group, an aryl group and an aryloxy group.

The alkenyl group referred in the present application may be linear, branched, cyclic, benzocyclic, or naphthocyclic and a linear or branched alkenyl group is preferred. The alkenyl group preferably has from 2 to 20 carbon atoms, more preferably from 2 to 12 carbon atoms, further preferably from 2 to 8 carbon atoms, still further preferably from 2 to 6 carbon atoms. Examples of the alkenyl group include a vinyl group, a butadienyl group, a hexatrienyl group, a 1-cyclohexenyl group. The cyclic group may be substituted or a fused ring. Examples of the cyclic group include thiophenyl, furanyl, dithiophenyl, pyrrolyl groups. The alkenyl group may be substituted. Examples of the substituent in this case include an alkyl, alkoxy group, an aryl group, an aryloxy group, an acyl group, a hydroxyl group, a halogen atom, a diarylamino group (including a 9-carbazolyl group) and a cyano group.

The aryl group referred in the present application may have a structure containing only one aromatic ring or a structure containing two or more aromatic rings condensed with each other. The aryl group preferably has from 6 to 22 ring skeleton-forming carbon atoms, more preferably from 6 to 18 ring skeleton-forming carbon atoms, further preferably from 6 to 14 ring skeleton-forming carbon atoms, and still further preferably from 6 to 10 ring skeleton-forming carbon atoms. Examples of the aryl group include a phenyl group, a 1-naphthyl group, a 2-naphthyl group, a 1-anthranyl group, a 2-anthranyl group, a 9-anthranyl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 1-pyrenyl group and a 2-pyrenyl group. The aryl group may be substituted. Examples of the substituent in this case include an alkyl group, an alkoxy group, an aryl group, an aryloxy group, an acyl group, a hydroxyl group, a halogen atom, a diarylamino group (including a 9-carbazolyl group) and a cyano group, and preferred are an alkyl group, an alkoxy group, an aryl group, and an aryloxy group.

All of the ring skeleton-forming atoms of the aryl group referred in the present application may be carbon atoms. The aryl group referred in the present application may be a heteroaryl group. The heteroaryl group referred in the present application may have a structure containing only one heteroaromatic ring or a structure containing two or more heteroaromatic rings condensed with each other. The heteroaryl group may contain at least one heteroaromatic ring and at least one aromatic ring. The heteroaryl group preferably has from 5 to 22 ring skeleton-forming atoms, more preferably from 5 to 18 ring skeleton-forming atoms, further preferably from 5 to 14 ring skeleton-forming atoms, and still further preferably from 5 to 10 ring skeleton-forming atoms. Examples of the heteroaryl group include a 2-thienyl group, a 3-thienyl group, a 2-furyl group, a 3-furyl group, a 2-pyrrolyl group, a 2-pyridyl group, a 3-pyridyl group, a 4-pyridyl group, a 2-pyrazinyl group, a 2-quinolyl group, a 3-quinolyl group, a 4-quinolyl group, a 1-isoquinolyl group and a 3-isoquinolyl group. Other examples of the heteroaryl group include a benzofuryl group, a pyrrolyl group, an indolyl group, an isoindolyl group, an azaindolyl group, a benzothienyl group, a pyridyl group, a quinolinyl group, an isoquinolyl group, an imidazolyl group, a benzimidazolyl group, a pyrazolyl group, an oxazolyl group, an isoxazolyl group, a benzoxazolyl group, a thiazolyl group, a benzothiazolyl group, an isothiazolyl group, a pyridazinyl group, a pyrimidinyl group, a pyrazinyl group, a triazinyl group, a cinnolinyl group, a phthalazinyl group and a quinazolinyl group. The heteroaryl group may be substituted. Examples of the substituent in this case include an alkyl group, an alkoxy group, an aryl group, an aryloxy group, a hydroxyl group, a halogen atom, a diarylamino group (including a 9-carbazolyl group) and a cyano group, and preferred are an alkyl group, an alkoxy group, an aryl group, and an aryloxy group.

For the alkyl moiety of the alkoxy group and the alkylthio group referred in the present application, reference may be made to the description for the alkyl group.

For the aryl moiety of the aryloxy group, the arylthiol group and diarylamino group referred in the present application, reference may be made to the description for the aryl group.

The halogen atom referred in the present application is preferably a fluorine atom, a chlorine atom, a bromine atom or an iodine atom.

The substituent referred in the present application may be an atom other than a hydrogen atom or a group having two or more atoms. Examples of the substituent include an alkyl group, an alkoxy group, an alkylthio group, an aryl group, an aryloxy group, an arylthiol group, a hydroxyl group, a halogen atom, a diarylamino group (including a 9-carbazolyl group), a cyano group and a combination thereof. The substituent may be selected from the group consisting of an alkyl group, an alkoxy group, an aryl group, an aryloxy group and a combination thereof; the group consisting of an alkyl group, an aryl group and a combination thereof; the group consisting of an aryl group, an aryloxy group and a combination thereof; the group consisting of an alkyl group, an alkoxy group and a combination thereof; or an alkyl group.

The compound of the invention is represented by the following formula (1):

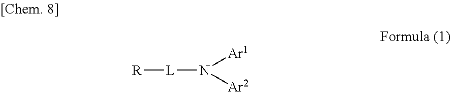

In the formula (1), Ar¹ and Ar² each independently represent a substituted or unsubstituted aryl group, and Ar¹ and Ar² may be bonded to each other to form a tri- or more-cyclic structure. Ar¹ and Ar² may be bonded via a direct bond to form for example a substituted or unsubstituted 9-carbazoryl group. Ar¹ and Ar² may be also bonded via a divalent group such as >C(R⁷)(R⁸), —O—, —S— or >N(R⁹). R⁷ to R⁹ each independently represent a hydrogen atom or a substituent.

In the formula (1), L represents a divalent group having at least one phenyl ring. The number of phenyl rings in L is preferably 1 to 15, more preferably 1 to 12, still more preferably 1 to 9. L may be selected from the divalent groups having 1 to 8 phenyl rings, the divalent groups having 1 to 5 phenyl rings, the divalent groups having 1 to 15 phenyl rings, the divalent groups having 7 to 15 phenyl rings. The phenyl rings may be those included in a group represented by the formula (2) or (4) below.

The divalent group for L consists of at least one group represented by the formula (2) below, optionally at least one group represented by the formula (3) below, and optionally at least one group represented by the formula (4) below:

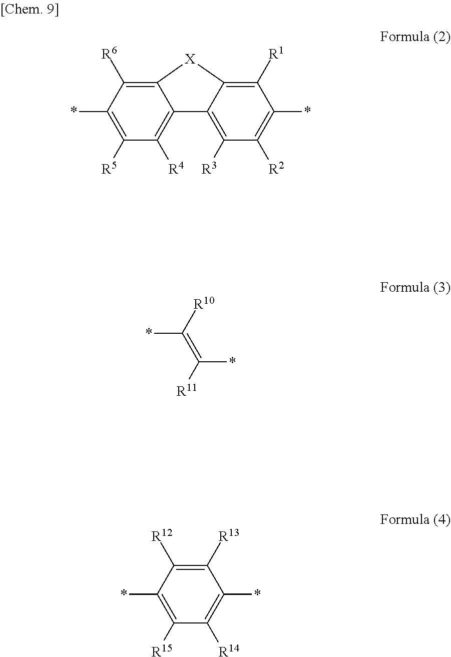

In the formulae (2) to (4), Each * represents a bonding site. X represents >C(R⁷)(R⁸), —O—, —S— or >N(R⁹). X may be selected from the group consisting of >C(R⁷)(R⁸), —S— and >N(R⁹), or the group consisting of >C(R⁷)(R⁸) and >N(R⁹). R¹ to R¹⁵ each independently represent a hydrogen atom or a substituent. R² and R³, R⁴ and R⁵, R¹² and R¹³ and R¹⁴ and R¹⁵ may be taken together to form a ring. The formed ring may have 4 to 10 ring skeleton-forming atoms, more preferably 5 to 8 ring skeleton-forming atoms, further preferably 5 to 7 ring skeleton-forming atoms. The formed ring may be an aliphatic ring and an aromatic ring. Examples of the ring include a cyclopentane ring, a cyclohexane ring, a cycloheptane ring, a phenyl ring. These rings may be substituted or unsubstituted and may be fused by at least one ring. R¹ to R⁶ and R¹⁰ to R¹⁵ may be a hydrogen atom.

The divalent group as L essentially has at least one group represented by the formula (2) and may or may not have at least one group represented by the formula (3) or (4). The divalent group as L may consist of one or more groups represented by the formula (2) only; at least one group represented by the formula (2) and at least one group represented by the formula (3); at least one group represented by the formula (2) and at least one group represented by the formula (4); or at least one group represented by the formula (2), at least one group represented by the formula (3) and at least one group represented by the formula (4). The number of the groups of formulae (2) to (4) which are linked to form the divalent group as L may be within the range of 2 to 20, the range of 2 to 15, the range of 2 to 10, or the range of 2 to 8. The number may be within the range of 3 to 20, the range of 4 to 20, the range of 5 to 20, or the range of 7 to 20. The divalent group as L may have a symmetric structure.

The divalent group as L may have a [formula (2)]-[formula (3)] unit, or a [formula (3)]-[formula (4)] unit. The divalent group as L may have a [formula (2)]-[formula (3)]-[formula (4)] unit. The divalent group as L may have a [formula (2)]-[formula (2)] unit, or a [formula (2)]-[formula (2)]-[formula (2)] unit. Examples of the divalent group as L include [formula (2)]-[formula (3)]-[formula (4)], [formula (4)]-[formula (3)]-[formula (2)], [formula (2)]-[formula (3)]-[formula (2)], [formula (4)]-[formula (2)]-[formula (2)], [formula (2)]-[formula (4)]-[formula (2)], [formula (2)]-[formula (2)]-[formula (4)], [formula (2)]-[formula (4)]-[formula (4)], [formula (4)]-[formula (2)]-[formula (4)], [formula (4)]-[formula (4)]-[formula (2)], [formula (4)]-[formula (4)]-[formula (4)], [formula (4)]-[formula (4)], [formula (4)]-[formula (2)] and [formula (2)]-[formula (4)]. Other examples of the divalent group as L include [formula (4)]-[formula (3)]-[formula (2)]-[formula (3)]-[formula (4)], [formula (4)]-[formula (3)]-[formula (2)]-[formula (2)]-[formula (3)]-[formula (4)], [formula (4)]-[formula (3)]-[formula (2)]-[formula (2)]-[formula (2)]-[formula (3)]-[formula (4)], [formula (4)]-[formula (3)]-[formula (2)]-[formula (2)]-[formula (2)]-[formula (2)]-[formula (3)]-[formula (4)], [formula (2)]-[formula (3)]-[formula (2)]-[formula (3)]-[formula (2)], [formula (2)]-[formula (3)]-[formula (2)]-[formula (2)]-[formula (3)]-[formula (2)], and [formula (2)]-[formula (3)]-[formula (2)]-[formula (2)]-[formula (2)]-[formula (3)]-[formula (2)]. Two or more groups represented by the formula (2), (3) or (4) included in the divalent group as L may be the same or different.

In the formula (1), R represents a hydrogen atom or a group represented by the following formula (5):

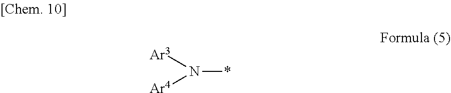

In the formula (5), Ar³ and Ar⁴ each independently represent a substituted or unsubstituted aryl group, and Ar³ and Ar⁴ may be bonded to each other to form a tri- or more-cyclic structure. Ar³ and Ar⁴ may be bonded via a direct bond to form for example a substituted or unsubstituted 9-carbazoryl group. Ar³ and Ar⁴ may be also bonded via a divalent group such as >C(R⁷)(R⁸), —O—, —S— or >N(R⁹). R⁷ to R⁹ each independently represent a hydrogen atom or a substituent. When R is a group represented by the formula (5), Ar³ and Ar⁴ may be the same as Ar¹ and Ar² in the formula (1), respectively, and the compound represented by the formula (1) may have a symmetric structure.

At least one alkyl group having at least five carbon atoms which are bonded is present in the formula (1). The alkyl group having at least five carbon atoms which are bonded may be straight or branched. The number of carbon atoms which are bonded in such an alkyl group may be within the range of 6 to 20, the range of 6 to 15, the range of 6 to 12, the range of 6 to 10. The number of carbon atoms which are bonded in such an alkyl group may be within the range of 8 to 20, the range of 8 to 15, the range of 8 to 12, the range of 8 to 10. Examples of the alkyl group having at least five carbon atoms which are bonded include a pentyl group, a n-hexyl group, a n-heptyl group, a n-octyl group, a n-nonyl group, a n-decyl group, a n-undecyl group, a n-dodecyl group, a n-tridecyl group, an isopentyl group, an isohexyl group, an isoheptyl group, an isooctyl group, an isononyl group, an isodecyl group, an isoundecyl group, an isododecyl group, an isotridecyl group and 2-ethylhexyl.

The number of such an alkyl group in the formula (1) may be within the range of 1 to 10, the range of 1 to 5, the range of 1 to 3. The number of such an alkyl group in the formula (1) may be 2 or more, within the range of 2 to 10, the range of 2 to 5. When the number of such an alkyl group in the formula (1) is 2 or more, they may be the same or different. The formula (1) may also have an alkyl group having less than five carbon atoms which are bonded.

An alkyl group having at least five carbon atoms which are bonded may be included in R, L, Ar¹ or Ar² in the formula (1) and Ar³ or Ar⁴ in the formula (5). R¹ to R¹⁵ in the formulae (2) to (4) may be an alkyl group having at least five carbon atoms which are bonded. When X in the formula (2) is >C(R⁷)(R⁸), at least one of R⁷ and R⁸ is preferably an alkyl group having at least five carbon atoms which are bonded.

The compound represented by the formula (1) may be a compound represented by the following formula (6):

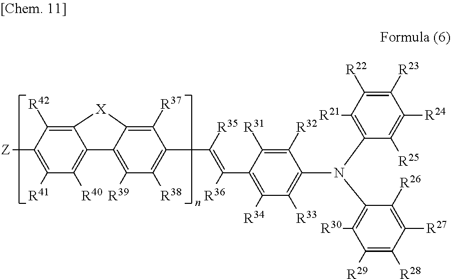

In the formula (6), X represents >C(R⁷)(R⁸), —O—, —S— or >N(R⁹). R⁷ to R⁹, R²¹ to R⁴² each independently represent a hydrogen atom or a substituent. R²¹ and R²², R²² and R²³, R²³ and R²⁴, R²⁴ and R²⁵, R²⁵ and R²⁶, R²⁶ and R²⁷, R²⁷ and R²⁸, R²⁸ and R²⁹, R²⁹ and R³⁰, R³¹ and R³², R³³ and R³⁴, R³⁸ and R³⁹, and R⁴⁰ and R⁴¹ may be taken together to form a ring. For R⁷ to R⁹ of X in the formula (6), reference may be made to the description for R⁷ to R⁹ of X in the formula (2). For R²¹ to R⁴² in the formula (6), reference may be made to the description for R¹ to R¹⁵ in the formulae (2) to (4).

In the formula (6), n is an integer of 1 to 10. n may be an integer of 1 to 6, an integer of 1 to 5, an integer of 1 to 4 or an integer of 1 to 3. When n is 2 or more, each of R³⁷ to R⁴² may be the same or different. All of R³⁷ to R⁴² may be a hydrogen atom.

In the formula (6), Z represents a hydrogen atom or a substituent. Examples of the substituents as Z include an alkoxy group, an aryl group, an aryloxy group, an acyl group, an alkenyl group, a hydroxyl group, a halogen atom, a diarylamino group (including a 9-carbazolyl group), a cyano group and a combination thereof. Z may be selected from the group consisting of a hydrogen atom, an alkoxy group, an aryl group, an aryloxy group, an alkenyl group, a diarylamino group (including a 9-carbazolyl group) and a combination thereof. Z may be selected from the group consisting of a hydrogen atom, an aryl group, an alkenyl group, a diarylamino group (including a 9-carbazolyl group) and a combination thereof. Z may be a hydrogen atom or a group represented by the following formula (7):

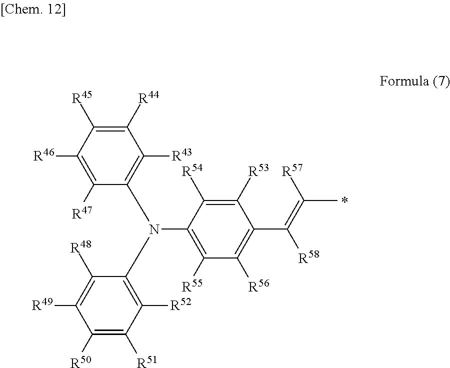

In the formula (7), R⁴³ to R⁵⁸ each independently represent a hydrogen atom or a substituent. R⁴³ and R⁴⁴, R⁴⁴ and R⁴⁵, R⁴⁵ and R⁴⁶, R⁴⁶ and R⁴⁷, R⁴⁷ and R⁴⁸, R⁴⁸ and R⁴⁹, R⁴⁹ and R⁵⁰, R⁵⁰ and R⁵¹, R⁵¹ and R⁵², R⁵³ and R⁵⁴, and R⁵⁵ and R⁵⁶ may be taken together to form a ring. * represents a bonding site. For R⁴³ to R⁵¹ in the formula (7), reference may be made to the description for R²¹ to R³⁶ in the formula (6) and the description of R¹ to R⁶ and R¹⁰ to R¹⁵ in the formulae (2) to (4). R⁴³ to R⁵¹ in the formula (7) may be the same as R²¹ to R³⁶ in the formula (6), respectively. All of R⁴³ to R⁵¹ may be a hydrogen atom. The compound represented by the formula (6) may have a symmetric structure.

Specific examples of the compounds represented by the formula (1) shown below. However, the compounds represented by the formula (1) capable of being used in the invention are not limited to the specific examples.

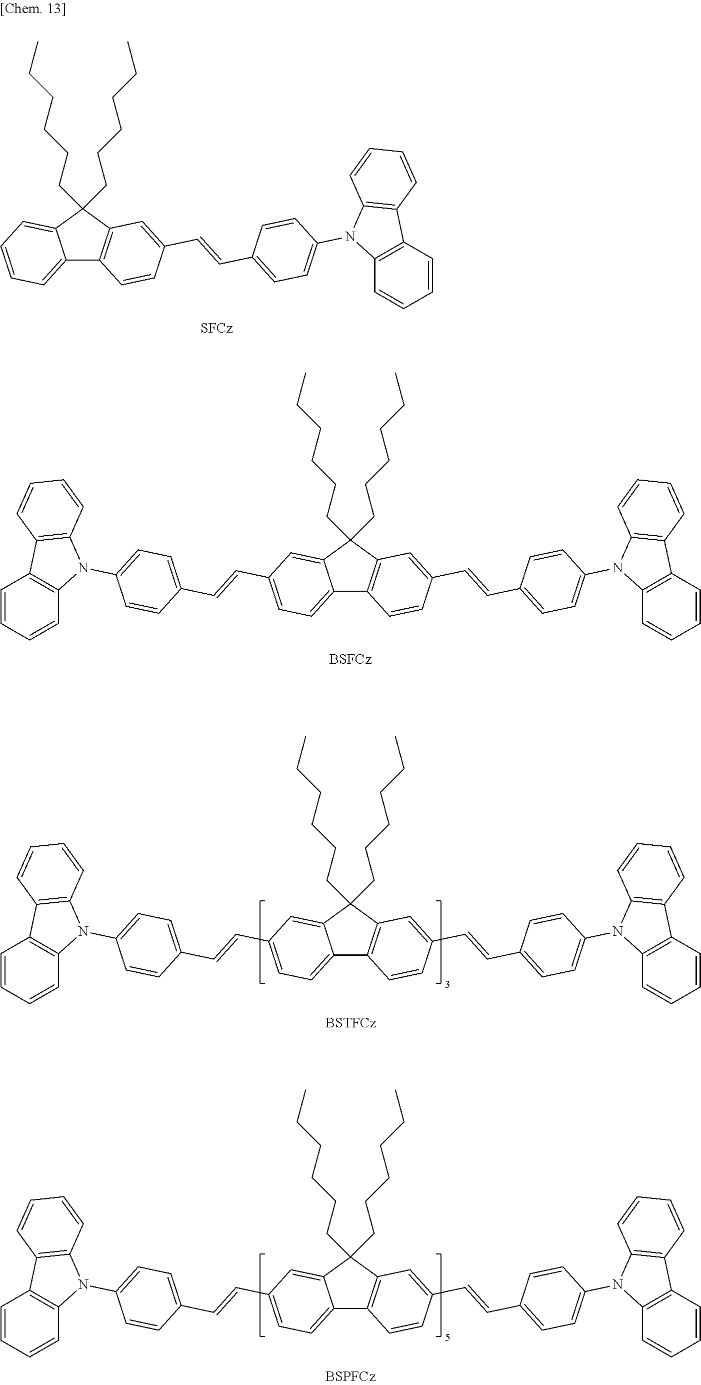

The compounds represented by the formula (1) can be synthesized by known reactions. For the details of the reactions, reference may be made to the synthesis examples described later.

This invention also provides an organic semiconductor laser containing a compound represented by the formula (1). A compound of the formula (1) is useful as a material used in a light-emitting layer (light amplification layer) of the organic semiconductor laser. The light-emitting layer may contain two or more compounds of the formula (1) but preferably contains only one compound of the formula (1). The light-emitting layer may contain a host material. Preferable host material absorbs photo-excitation light for the organic semiconductor laser. Another preferable host material has sufficient spectral overlap between its fluorescence spectrum and the absorption spectrum of the compound of the formula (1) contained in the light-emitting layer so that an effective Forster-type energy transfer can take place from the host material to the compound of the formula (1). The concentration of the compound of the formula (1) in the light-emitting layer is preferably at least 0.1 wt %, more preferably at least 1 wt %, still more preferably at least 3 wt %, and preferably at most 50 wt %, more preferably at most 30 wt %, still more preferably at most 10 wt %.

The organic semiconductor laser of this invention has an optical resonator structure. The optical resonator structure may be a one-dimensional resonator structure or a two-dimensional resonator structure. Examples of the latter include a circulator resonator structure, and a whispering gallery type optical resonator structure. A distributed feedback (DFB) structure and a distributed Bragg reflector (DBR) structure are also employable. For DFB, a mixed-order DFB grating structure is preferably employed. Namely, a mixed structure of DFB grating structures differing in point of the order relative to laser emission wavelength may be preferably employed. Specific examples thereof include an optical resonator structure composed of a second-order Bragg scattering region surrounded by the first-order Bragg scattering region and a mixed structure where a second-order Bragg scattering region and a first-order scattering region are formed alternately. For details of preferred optical resonator structures, specific examples to be given hereinunder may be referred to. As the optical resonator structure, the organic semiconductor laser may be further provided with an external optical resonator structure. For example, the optical resonator structure may be formed preferably on a glass substrate. The material to constitute the optical resonator structure includes an insulating material such as SiO₂, etc. For example, a grating structure is formed, the depth of the grating is preferably 75 nm or less, and is more preferably selected from a range of 10 to 75 nm. The depth may be, for example, 40 nm or more, or may be less than 40 nm. The light-emitting layer (light amplification layer) containing a compound of the formula (1) can be directly formed on the optical resonator structure.

The organic semiconductor laser is preferably encapsulated by a sapphire or other materials to lower the lasing threshold and optimize the heat dissipation under intense optical pumping. An interlayer may be formed between the sapphire lid and the light-emitting layer. For example, amorphous fluorinated polymer such as CYTOP (trademark) is preferably used in the interlayer.

Other advantages and features of this invention may be better understood with respect to the following examples given for illustrative purposes and the accompanying figures.

### EXAMPLES

The invention will be described more specifically with reference to synthesis examples and working examples below. The materials, processes, procedures and the like shown below may be appropriately modified unless they deviate from the substance of the invention. Accordingly, the scope of the invention is not construed as being limited to the specific examples shown below.

### Syntheses

Three compounds SFCz, BSFCz and BSTFCz were synthesized by the following scheme:

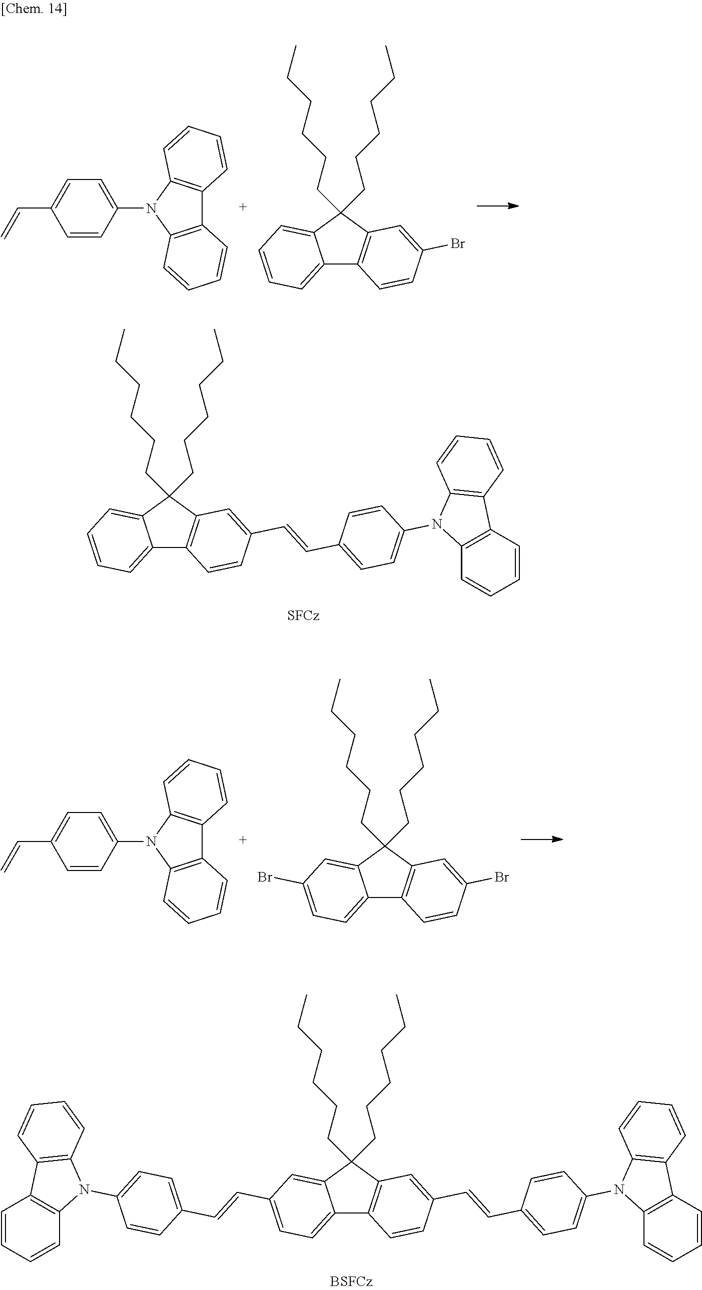

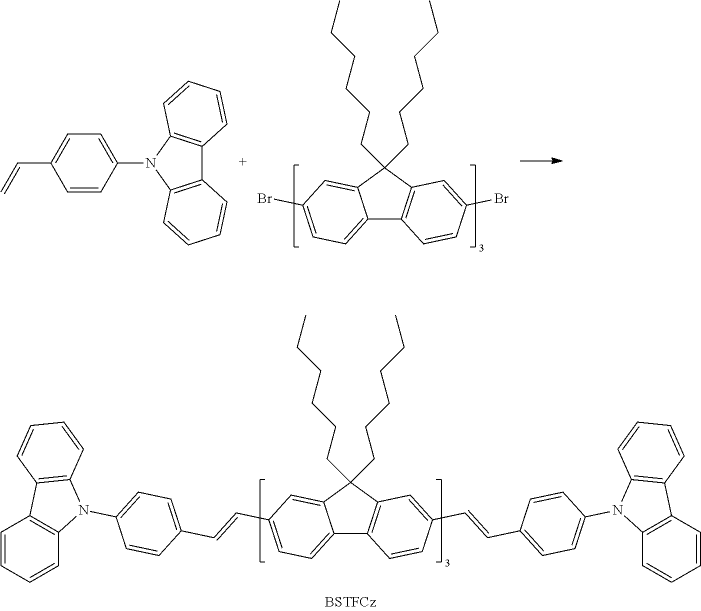

### Synthesis Example 1

### (E)-9-(4-(2-(9,9-dihexyl-9H-fluoren-2-yl)vinyl)phenyl)-9H-carbazole, SFCz

A mixture of 9-(4-Vinylphenyl)-9H-carbazole (383 mg, 1.42 mmol), 2-Bromo-9,9-dihexyl-9H-fluorene (531 mg, 1.29 mmol), tri(o-tolyl)phosphine (34.9 mg, 0.115 mmol), palladium(II) acetate (8.0 mg, 0.036 mmol) and triethylamine (3.0 mL) was dissolved in anhydrous dimethylformamide (6.0 mL). The solution was quickly deoxygenated under vacuum and back-filled with Ar gas. This process was repeated 3 times. The reaction mixture was then stirred in a 90° C. oil bath under Ar gas for 4 hours. The mixture was cooled to room temperature. Water (50 mL) and diethyl ether (50 mL) were added to the mixture and the two layers were separated. The aqueous layer was extracted with diethyl ether (2×40 mL). All organic layers were combined, washed with water (3×70 mL), dried over anhydrous magnesium sulfate and filtered. The filtrate was collected and solvent removed under reduced pressure to give a yellow gummy solid. The crude was purified by column chromatography over silica using dichloromethane/petroleum (1:8) as eluent to give SFCz as a white solid (429 mg, 55%); m.p.: 120.2-121.6° C.; T_{d}(5%)=395° C.; vₘₐₓ(solid)/cm⁻¹: 723 (vs), 750 (vs), 831 (s), 965 (s), 1228 (s), 1451 (vs), 1516 (s), 2854 (m), 2927 (m), 2937 (w), 3053 (w); λₘₐₓ (dichloromethane)/nm: 236 (log ε/dm³ mol⁻¹ cm⁻¹ 4.78), 258 sh (4.43), 286 sh (4.28), 294 (4.41), 356 (4.77). ¹H NMR (500 MHz, CDCl₃) δ 0.62-0.72 (4H, m, CH₂), 0.78 (6H, t, J=7.0, CH₃), 1.03-1.16 (12H, m, CH₂), 2.00-2.03 (4H, m, Fl-CH₂), 7.25-7.37 (7H, m, Cz-H, CH═CH & Fl-H), 7.42-7.48 (4H, m, Cz-H), 7.53 (1H, s, Fl-H), 7.54-7.60 (3H, m, Fl-H & Ph-H), 7.70-7.72 (2H, m, Fl-H), 7.78 (2H, 1/2AA′XX′, Ph-H), 8.15-8.17 (2H, m, Cz-H); ¹³C NMR (125 MHz, CDCl₃) δ 14.0, 22.6, 23.8, 29.8, 31.5, 40.5, 55.1, 109.9, 119.7, 119.9, 120.0, 120.3, 120.9, 122.9, 123.4, 125.7, 126.0, 126.8, 127.15, 127.24, 127.7, 130.3, 136.0, 136.7, 136.8, 140.7, 140.8, 141.3, 151.0, 151.4; m/z (ESI): calculated for C₄₅H₄₇N [M]: 601.4 (100%), 602.4 (49%), 603.4 (12%); found C₄₅H₄₇N [M]: 601.4 (100%), 602.4 (49%), 603.4 (12%); C₄₅H₄₇N requires C, 89.80; H, 7.87; N, 2.33; found: C, 89.63; H, 7.57; N, 2.30%.

### Synthesis Example 2

### 9,9′-(((1E,1′E)-(9,9-dihexyl-9H-fluorene-2,7-diyl)bis(ethene-2,1-diyl))bis(4,1-phenylene))bis(9H-carbazole), BSFCz

A mixture of 9-(4-Vinylphenyl)-9H-carbazole (700 mg, 2.60 mmol), 2,7-Dibromo-9,9-dihexyl-9H-fluorene (510 mg, 1.04 mmol), tri(o-tolyl)phosphine (37.7 mg, 0.124 mmol), palladium(II) acetate (7.0 mg, 0.031 mmol) and triethylamine (4.0 mL) was dissolved in anhydrous dimethylformamide (11 mL). The solution was quickly deoxygenated under vacuum and back-filled with Ar gas. This process was repeated 3 times. The reaction mixture was then stirred in a 90° C. oil bath under Ar gas for 4 hours. The mixture was cooled to room temperature. Water (100 mL) and diethyl ether (100 mL) were added to the mixture and the two layers were separated. The aqueous layer was extracted with diethyl ether (100 mL). All organic layers were combined, washed with water (3×150 mL), dried over anhydrous magnesium sulfate and filtered. The filtrate was collected and solvent removed under reduced pressure. The crude was purified by column chromatography over silica using dichloromethane/petroleum (1:7) as eluent to give BSFCz as a light yellow solid (340 mg, 38%); m.p.: 175.3-192.8° C.; T_{d}(5%)=436° C.; ¹H NMR (500 MHz, CDCl₃) δ 0.70-0.76 (4H, m, CH₂), 0.79 (6H, t, J=6.5, CH₃), 1.06-1.17 (12H, m, CH₂), 2.06-2.09 (4H, m, Fl-CH₂), 7.29-7.35 (8H, m, Cz-H & CH═CH), 7.41-7.49 (8H, m, Cz-H), 7.54 (2H, s, Fl-H), 7.56-7.61 (6H, m, Fl-H & Ph-H), 7.73 (2H, d, J=8.0, Fl-H), 7.79- (4H, 1/2AA′XX′, Ph-H), 8.15-8.18 (4H, m, Cz-H); ¹³C NMR (125 MHz, CDCl₃) δ 14.0, 22.6, 23.8, 29.8, 31.5, 40.5, 55.1, 109.8, 119.97, 120.04, 120.3, 120.9, 123.4, 125.8, 125.9, 126.9, 127.2, 127.7, 130.2, 136.1, 136.7, 140.8, 140.9, 151.7; vₘₐₓ(solid)/cm⁻¹: 722 (vs), 746 (vs), 825 (s), 960 (s), 1226 (s), 1451 (vs), 1514 (s), 1597 (m), 2849 (w), 2923 (m), 2964 (w); λₘₐₓ (dichloromethane)/nm: 239 (log ε/dm³ mol⁻¹ cm⁻¹ 4.99), 257 sh (4.79), 287 sh (4.49), 292 (4.56), 332 sh (4.40), 387 (5.02), 409 sh (4.91); m/z (ESI): calculated for C₆₅H₆₀N₂ [M]: 868.5 (100%), 869.5 (70%), 870.5 (24%); found C₆₅H₆₀N₂ [M]: 868.5 (100%), 869.5 (74%), 870.5 (26%); C₆₅H₆₀N₂ requires C, 89.82; H, 6.96; N, 3.22; found: C, 89.79; H, 6.91; N, 3.22%. The large range of recorded melting point can be explained by the presence of multiple morphological solids with different melting points, and is consistent with observations from DSC.

### Synthesis Example 3

### 9,9′-(((1E,1′E)-(9,9,9′,9′,9″,9″-hexahexyl-9H,9′H,9″H-[2,2′:7′,2″-terfluorene]-7,7″-diyl)bis(ethene-2,1-diyl))bis(4,1-phenylene))bis(9H-carbazole), BSTFCz

A mixture of 9-(4-Vinylphenyl)-9H-carbazole (603 mg, 2.24 mmol), 7,7″-Dibromo-9,9,9′,9′,9″,9″-hexahexyl-9H,9′H,9″H-2,2′:7′,2″-terfluorene (1.04 g, 0.897 mmol), tri(o-tolyl)phosphine (42.4 mg, 0.139 mmol), palladium(II) acetate (11.0 mg, 0.049 mmol) and triethylamine (5.0 mL) was dissolved in anhydrous dimethylformamide (22 mL). The solution was quickly deoxygenated under vacuum and back-filled with Ar gas. This process was repeated 3 times. The reaction mixture was then stirred in a 90° C. oil bath under Ar gas for 4 hours. The mixture was cooled to room temperature. Water (100 mL) and diethyl ether (100 mL) were added to the mixture and the two layers were separated. The aqueous layer was extracted with diethyl ether (70 mL). All organic layers were combined, washed with water (3×150 mL), dried over anhydrous magnesium sulfate and filtered. The filtrate was collected and solvent removed under reduced pressure to give a viscous yellow oil. The crude was purified by column chromatography over silica using dichloromethane/petroleum (1:7) as eluent to give BSTFCz as a light yellow solid (536 mg, 39%); m.p.: 119.1-120.8° C.; T_{d}(5%)=433° C.; ¹H NMR (500 MHz, CDCl₃) δ 0.77-0.87 (30H, m, CH₂ & CH₃), 1.10-1.19 (36H, m, CH₂), 2.08-2.15 (12H, m, Fl-CH₂), 7.28-7.36 (8H, m, Cz-H & CH═CH), 7.42-7.50 (8H, m, Cz-H), 7.57-7.62 (8H, m, Fl-H & Ph-H), 7.65-7.71 (8H, m, Fl-H), 7.76-7.86 (10H, m, Fl-H & Ph-H), 8.17 (4H, m, Cz-H); ¹³C NMR (125 MHz, CDCl₃) δ 14.0, 22.5, 22.6, 23.8, 29.6, 29.7, 31.4, 31.5, 40.3, 40.4, 55.2, 55.3, 109.8, 119.97, 120.02, 120.3, 120.9, 121.4, 121.5, 123.4, 125.8, 125.9, 126.2, 126.9, 127.2, 127.7, 130.3, 136.0, 136.7, 136.8, 139.9, 140.0, 140.5, 140.6, 140.8, 140.9, 151.7, 151.8; vₘₐₓ(solid)/cm⁻¹: 722 (vs), 746 (vs), 817 (s), 961 (m), 1228 (s), 1451 (vs), 1514 (s), 2852 (m), 2925 (m), 2969 (w); λₘₐₓ (dichloromethane)/nm: 237 (log ε/dm³ mol⁻¹ cm⁻¹ 5.14), 257 sh (4.85), 286 sh (4.60), 293 (4.69), 326 sh (4.60), 385 (5.30), 397 sh (5.29); m/z (ESI): calculated for C₁₁₅H₁₂₄N₂[M]: 1533.0 (80%), 1534.0 (100%), 1535.0 (62%), 1536.0 (25%); found C₁₁₅H₁₂₄N₂[M]: 1533.0 (73%), 1534.0 (100%), 1535.0 (67%), 1536.0 (29%); C₁₁₅H₁₂₄N₂ requires C, 90.03; H, 8.15; N, 1.83; found: C, 89.99; H, 8.14; N, 1.82%.

Thermal Properties

Thermal properties of the new chromophores were studied using thermogravimetric analysis (TGA) and differential scanning calorimetry (DSC) under nitrogen atmosphere. All three chromophores showed high thermal stability with decomposition temperatures (T_{d}, referring to 5% weight loss) at 395, 436 and 433° C. for SFCz, BSFCz and BSTFCz, respectively, which are comparable to that reported soluble BSB-Cz derivatives (M. Mamada, T. Fukunaga, F. Bencheikh, A. S. D. Sandanayaka, C. Adachi, Adv. Funct. Mater. 2018, 28, 1802130). SFCz, BSFCz and BSTFCz were found to have glass transition temperatures (T_{g}) at 49, 104 and 105° C., respectively. The results show that the inclusion of the 2^{nd} VPC moiety (i.e., moving from SFCz to BSFCz) improves their thermal property while the increase of the central fluorene unit numbers (i.e., moving from BSFCz to BSTFCz) has negligible effect on T_{g} and T_{d} values. The higher T_{g} of BSFCz than SFCz can be attributed to the increase of size of the material, while the similar T_{g} of BSTFCz with BSFCz is a trade result of the size and the flexible hexyl moieties attached to the molecule.

### Photophysical Properties

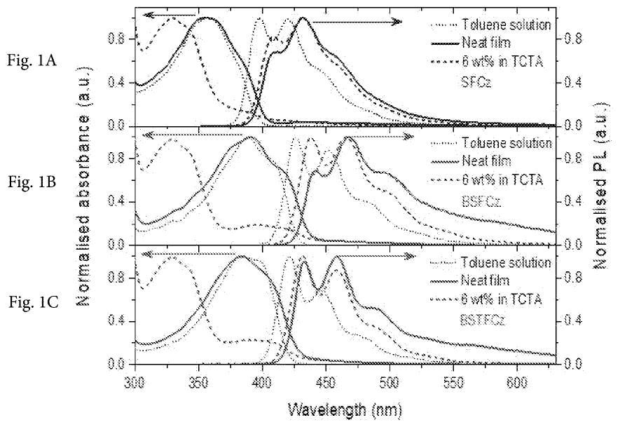

Photophysical properties of the new chromophores were probed in solution (dichloromethane and toluene), blend films (6 wt % in TCTA, spin-coated from chloroform solution) and neat films (spin-coated from chloroform solution). FIG. 1 shows the steady-state solution and film absorption, as well as photoluminescence (PL) spectra of the materials and the corresponding values are summarised in Table 1.

First, all three compounds exhibit high molar extinction coefficients (ε), which is desirable for efficient photoexcitation under optical pumping. Specifically, the corresponding F values at their absorption maxima were found 0.59×10⁵ dm³ mol⁻¹ cm⁻¹ (at 356 nm), 1.06×10⁵ dm³ mol⁻¹ cm⁻¹ (at 387 nm) and 1.98×10⁵ dm³ mol⁻¹ cm⁻¹ (at 385 nm) for SFCz, BSFCz and BSTFCz, respectively. These values indicate that the extension of r-conjugation both through the inclusion of the second VPC moiety (i.e. SFCz to BSFCz) and the extension of the central fluorene units (i.e. BSFCz to BSTFCz) significantly enhance their F values (with approximately tripled F for BSTFCz moving from its parent SFCz).

The inclusion of the second VPC end group (i.e., from SFCz to BSFCz) also results in the red-shifted of absorption by approximately 30 nm (FIG. 1 (a), (b) and Table 1) due to the extension of the effective π-conjugation in BSFCz, compared to SFCz. Interestingly, similar effect was not observed by the extension of the central fluorenyl moiety of BSFCz (one fluorene) to form BSTFCz (three fluorenes) to give essentially the same absorption maxima (FIG. 1 (a), (c)). This is likely due to the partial disruption of the effective π-conjugation in BSTFCz due to increased molecular flexibility caused by the extended central fluorene units.

In solution, all three compounds exhibited distinctive vibronic structures with their 0-0 vibronic transitions peaked at 397, 421 and 426 nm for SFCz, BSFCz and BSTFCz, respectively, (FIG. 1). While the trend of emission peak follows their absorption maxima, distinctive vibronic PL structures suggest weak extent of rotational freedom of the molecules. Moving from solution to neat films, red-shifts in both absorption and PL were observed (FIG. 1), indicating strong intermolecular interaction. Interestingly, the neat film PL of SFCz apparently did not show spectrum broadening, giving similar full-width-at-half-maxima (FWHM) as its solution. Both BSFCz and BSTFCz showed greater FWHM moving from solution to neat films, where BSFCz showed significantly intense excimer emission (than BSTFCz, which is likely due to slightly more 3-dimensional structure in BSTFCz than BSFCz due to the higher numbers of dihexyl groups attached to the three fluorenes).

All three chromophores show high solution PLQYs (in toluene) of 65%, 85% and 81% for SFCz, BSFCz and BSTFCz, respectively. Moving from solution to neat film, the PLQYs of SFCz, BSFCz and BSTFCz were 70%, 31% and 45%, respectively. The reduction in neat film PLQYs of BSFCz and BSTFCz can be attributed to concentration quenching effect, which agrees with their red-shift and broadening of their PL spectra (FIG. 1). This was further supported by their reduction in PL lifetimes [0.61 ns (BSFCz) and 0.56 ns (BSTFCz)] of neat films, compared to those [0.86 ns (BSFCz) and 0.66 ns (BSTFCz)] in solutions using time-correlated single photon counting (TCSPC) measurements. The relatively less reduction in neat film PLQY of BSTFCz than that of BSFCz can be attributed to the more three dimensional structure in nature of BSTFCz than BSFCz as noted earlier. In contrast, SFCz shows aggregate-induced luminescence enhancement (AIE) phenomenon as seen by the increased neat-film PLQY of 70%, compared to that (65%) in solution, accompanied by an increase in neat film lifetime (1.47 ns), compared to that (1.03 ns) in solution. We attributed this AIE effect in SFCz to the difference in its structure and thus different packing motif in solid state.

We also studied their photophysics of blended films (6 wt % in TCTA). All blend film PL spectra were red-shifted compared to those of their solution (FIG. 1). This red-shift in PL spectra of blend films is often an indication of aggregation emission and can often be circumvented by further reduction in doping percentage. However, concentration-dependent studies in BSFCz and BSTFCz blend films (4 wt %, 6 wt %, 8 wt % and 10 wt %) show no considerable change in their PL spectra, even at doping concentration as low as 2 wt %. Nonetheless, similar PLQYs values in blend films and diluted solutions (Table 1) suggest minimal concentration quenching effects caused by aggregates in these blend films.

With their excited-state lifetimes being determined using TCSPC, their radiative decay rates (kᵣ) were calculated based on PLQYs and lifetimes data (S.-C. Lo, C. P. Shipley, R. N. Bera, R. E. Harding, A. R. Cowley, P. L. Burn, I. D. W. Samuel, Chem. Mater. 2006, 18, 5119-5129). We found that the extension of π-conjugation from SFCz to BSFCz to BSTFCz indeed leads to the increase in kᵣ values of 0.63×10⁹ s⁻¹, 0.99×10⁹ s⁻¹, and 1.24×10⁹ s⁻¹, respectively. The kᵣ values of BSFCz and BSTFCz are comparable with those of state-of-the-art organic laser dyes such as octafluorene (1.7×10⁹ s⁻¹), BSBCz (1.0×10⁹ s⁻¹), and spiro-SBCz (1.16×10⁹ s⁻¹); and are essential to achieve low ASE thresholds (D. H. Kim, A. S. D. Sandanayaka, L. Zhao, D. Pitrat, J. C. Mulatier, T. Matsushima, C. Andraud, J. C. Ribierre, C. Adachi, Appl. Phys. Lett. 2017, 110, 023303; T. Aimono, Y. Kawamura, K. Goushi, H. Yamamoto, H. Sasabe, C. Adachi, Appl. Phys. Lett. 2005, 86, 071110; and H. Nakanotani, S. Akiyama, D. Ohnishi, M. Moriwake, M. Yahiro, T. Yoshihara, S. Tobita, C. Adachi, Adv. Funct. Mater. 2007, 17, 2328-2335). Moving from solution to neat films, kᵣ values of all three compounds SFCz, BSFCz and BSTFCz decreased by about 50-75%. However, by blending the chromophores in TCTA host, kᵣs of all blend films of the three chromophores are significantly improved and close to those in solution (Table 1).

TABLE 1
Photophysical properties of SFCz, BSFCz and BSTFCz in toluene
solution, blend (6 wt % in TCTA) and neat films, spin-coated from
chloroform solution.

| | | λ_{abs} (nm), | | | | |
|---|---|---|---|---|---|---|
| | | [ε dm³ | λ_{PL} | PLQY | lifetime | kᵣ |
| | | mol⁻¹ cm⁻¹] | (nm) | (%) | (ns) | (×10⁹ s⁻¹) |
| SFCz | Solution | 356 | 397 | 65 ± 3 | 1.03 | 0.63 |
| | | [0.59 × 10⁶] | | | | |
| | Blend film | 330 | 431 | 71 ± 8 | 1.40* | 0.51 |
| | Neat film | 358 | 432 | 70 ± 8 | 1.47 | 0.48 |
| BSFCz | Solution | 387 | 426 | 85 ± 4 | 0.86 | 0.99 |
| | | [1.06 × 10⁶] | | | | |
| | Blend film | 330 | 465 | 76 ± 4 | 0.81* | 0.94 |
| | Neat film | 390 | 468 | 31 ± 9 | 0.61 | 0.51 |
| BSTFCz | Solution | 385 | 421 | 81 ± 4 | 0.66 | 1.24 |
| | | [1.98 × 10⁶] | | | | |
| | Blend film | 329 | 431 | 80 ± 6 | 0.73 | 1.10 |
| | Neat film | 383 | 459 | 45 ± 9 | 0.56 | 0.81 |

*total lifetime.

### ASE Properties

Solid-state ASE studies were conducted with neat and blend films of SFCz, BSFCz and BSTFCz in order to evaluate their potential use as optical gain materials, where the films were spin-coated from chloroform solution (25 mg ml⁻¹). The estimated ASE Eₜₕ are summarised in Table 2.

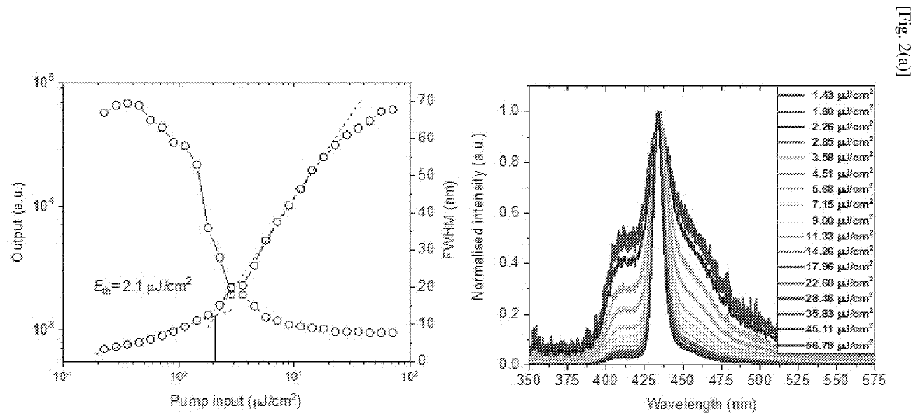

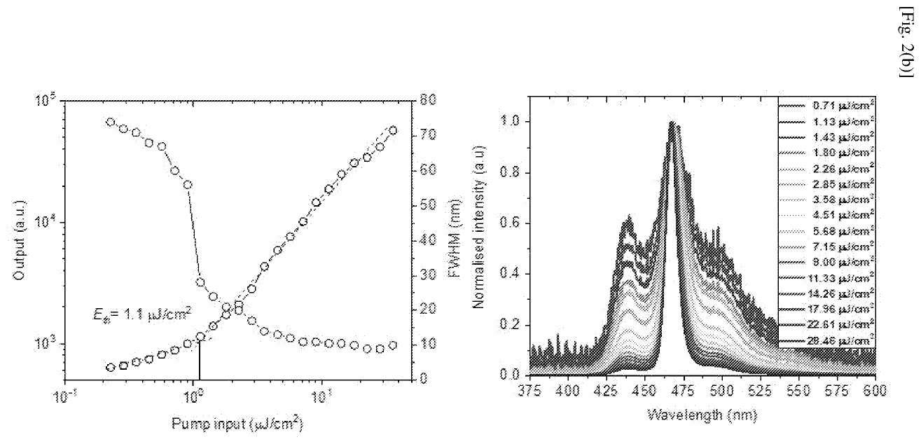

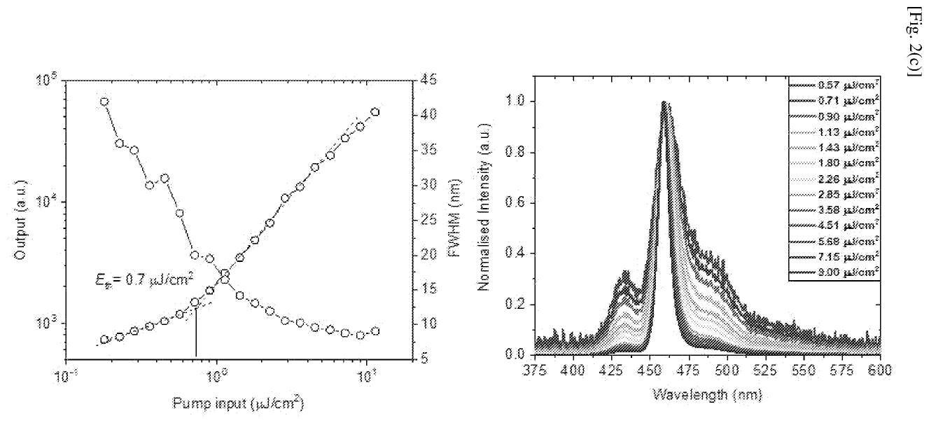

The ASE thresholds for solution-processed neat-film of all three chromophores were found to be low, ranging from 2.5 ρJ cm⁻² (for BSTFCz), to 4.4 ρJ cm⁻² (for BSFCz) and 5.5 ρJ cm⁻² (for SFCz). In-depth ASE studies were further conducted for solution-processed blend films of all 3 compounds at various doping concentrations in TCTA, a common host in OLEDs. While two doping concentration (6 wt % and 10 wt %) were measured for SFCz, more thorough ASE measurements at various doping concentration (2 wt %, 4 wt %, 6 wt %, 8 wt % and 10 wt %) were conducted for BSFCz and BSTFCz because of their superior F, high blend film PLQY, and more importantly, high kᵣ. All blend films of the three chromophores showed ASE Eₜₕ ranging from 0.7 to 2.1 J cm⁻² (Table 2). The lowest blend-film thresholds of SFCz, BSFCz and BSTFCz were determined to be 2.0±0.2, 1.1±0.2 and 0.7±0.1 ρJ cm⁻², respectively (FIG. 2(a)-FIG. 2(c)). Interestingly, the ASE Eₜₕ decreases as the π-conjugation is extended from SFCz to BSFCz to BSTFCz, which is consistent with the increase in both F and kᵣ values of the materials as noted earlier (Table 1). Accordingly, 6 wt % blend thin film of BSTFCz in TCTA showed the lowest threshold of 0.70 ρJ cm⁻², which is comparable to other current state-of-the-art organic semiconducting dyes.

ASE happened at the 0-1 vibrations for all the three chromophores (for both neat and various blend films) (FIG. 2). This phenomenon is common in organic laser dyes and can be explained by the presence of an efficient quasi-four energy level system at this transition.

TABLE 2
Solid-state ASE Eₜₕ (μJ cm⁻²) of SFCz, BSFCz and BSTFCz in blend
(at various doping concentrations) and neat films.

| | Blend ratio in TCTA | | | | | Neat |
|---|---|---|---|---|---|---|
| | 2 wt % | 4 wt % | 6 wt % | 8 wt % | 10 wt % | film |
| SFCz | — | — | 2.1 | — | 2.0 | 5.5 |
| BSFCz | 1.8 | 1.6 | 1.1 | 1.2 | 1.1 | 4.4 |
| BSTFCz | 1.0 | 0.8 | 0.7 | 0.75 | 0.85 | 2.5 |

### Transient Absorption Spectroscopy Measurements

To understand the dynamics and absorption features of singlet and triplet excited-states, nano-second transient absorption spectroscopy (TAS) measurements were conducted for SFCz, BSFCz and BSTFCz in toluene solution. All the samples showed the presence of long-lived and short-lived absorption features. These short-lived excited-state absorption and emission features (with negative differential absorption) have similar decay dynamics (1 ns for SFCz, 0.83 ns for BSFCz and 0.69 ns for BSTFCz) to the emission lifetimes (1.03 ns for SFCz, 0.86 ns for BSFCz and 0.66 ns for BSTFCz) obtained from the TCSPC measurements. Therefore, these short-lived absorption features are attributed to singlet excited-state absorptions while the long-lived features (0.13 μs for BSFCz and 0.15 μs for BSTFCz) are attributed to the triplet excited-state absorptions. The singlet excited-state emission, absorption and triplet excited-state absorption feature for SFCz, BSFCz and BSTFCz, respectively. The peak-to-peak intensity of singlet excited-state absorption was more than 20-time higher than triplet excited-state absorption. This low triplet yields were found in all three samples and are attributed to the high solution PLQYs of the materials.

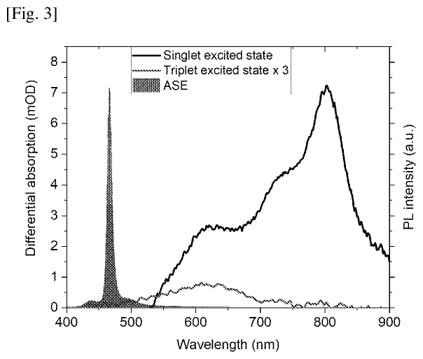

While both SFCz and BSTFCz show overlapping triplet absorption in gain region, BSFCz shows minimal triplet absorption at its ASE wavelength (FIG. 3). As mentioned earlier, this separation of triplet absorption from the gain region seen in BSFCz is highly desirable to progress toward long-pulse excitation (and ultimately organic laser diodes). This result prompted our further testings in CW lasing operation of BSFCz.

### Lasing Studies in DFB Structures Under CW Operation

In a DFB structure, a laser oscillation takes place when the following Bragg condition is satisfied: mλ_{Bragg}=²n_{eff}Λ, where m is the order of diffraction, λ_{Bragg} is the Bragg wavelength, n_{eff} is the effective refractive index of the gain medium, and Λ is the period of the grating. Here, mixed-order DFB resonator structure which comprises of second-order scattering regions surrounded by first-order scattering regions were incorporated in order to achieve low lasing threshold. Second-order DFB structure gives vertically out coupled, highly diverge beam with high lasing threshold due to radiation loss, while first-order DFB structure provides strong feedback and emits at the edge of the device, giving rise to low diverge beam with low lasing threshold. We incorporated a mixed-order DFB structure to introduce both features of first and second-order DFB resonator structures to obtain low lasing threshold. The grating periods (Λ) for mixed-order grating structure (m=1, 2) of each chromophores were calculated using λ_{ASE} as λ_{Bragg} and reported n_{eff} value (1.70) of a structurally similar chromophore BSBCz, and are shown in Table 3. The lengths of the individual first- and second-order DFB grating regions were 1.536, 1.656, 1.632 and 1.024, 1.104, 1.080 μm, respectively for SFCz, BSFCz and BSTFCz.

TABLE 3
Calculated period values (Λ) of mixed-order grating
structures for SFCz, BSFCz and BSTFCz.

| | Bragg | Period value | Period value |
|---|---|---|---|
| | wavelength | for m = 1 | for m = 2 |
| Molecule | (nm) | (nm) | (nm) |
| SFCz | 434 | 128 | 256 |
| BSFCz | 468 | 138 | 276 |
| BSTFCz | 460 | 136 | 272 |

Glass substrates (Atsugi Micro Co.) were cleaned by ultrasonication using neutral detergent, pure water, acetone, and isopropanol followed by UV-ozone treatment. A 100-nm-thick layer of SiO₂, which would become the DFB grating, was sputtered at 100° C. onto the glass substrates. The argon pressure during the sputtering was 0.66 Pa. The RF power was set at 100 W. Substrates were again cleaned by ultrasonication using isopropanol followed by UV-ozone treatment. The SiO₂ surfaces were treated with hexamethyldisilazane (HMDS) by spin coating at 4,000 rpm for 15 s and annealed at 120° C. for 120 s. A resist layer with a thickness of around 70 nm was spin-coated on the substrates at 4,000 rpm for 30 s from a ZEP520A-7 solution (ZEON Co.) and baked at 180° C. for 240 s.

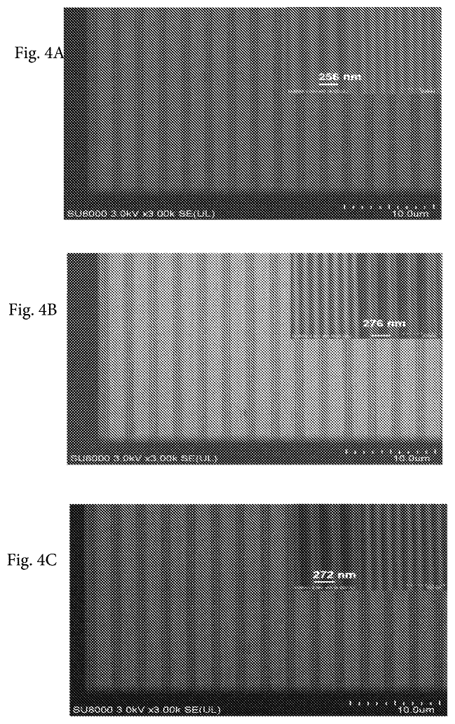

Electron beam lithography was performed to draw grating patterns on the resist layer using a JBX-5500SC system (JEOL) with an optimized dose of 0.1 nC cm⁻². After the electron beam irradiation, the patterns were developed in a developer solution (ZED-N50, ZEON Co.) at room temperature. The patterned resist layer was used as an etching mask while the substrate was plasma etched with CHF₃ using an EIS-200ERT etching system (ELIONIX). To completely remove the resist layer from the substrate, the substrate was plasma-etched with O₂ using a FA-1EA etching system (SAMCO). The area of a resonator structure was 5×5 mm² and lengths of first-order grating and second-order grating were designed as shown in Table 3 for each chromophore. The gratings formed on the SiO₂ surfaces were observed with SEM (SU8000, Hitachi) (FIG. 4). The DFB gratings fabricated in this work had grating periods value as shown in Table 3 with a grating depth of about 65±5 nm. To complete the laser devices, 200-nm-thick 6 wt % BSFCz: TCTA blend film, 6 wt % SFCz: TCTA blend film, 6 wt % BSTFCz: TCTA blend film, BSFCz neat film, SFCz neat film and BSTFCz neat films were prepared on the gratings by spin-coating at 2500 rpm for 60 s. Finally, 0.05 ml of CYTOP (Asahi Glass Co. Ltd., Japan) was directly spin-coated at 1000 rpm for 30 s onto the DFB laser devices, sandwiched with sapphire lids to seal the top of the laser devices, and dried under vacuum for 12 hours.

For the characterization of the pulsed organic lasers, pulsed excitation light from a nitrogen-gas laser (SRS, NL-100) was focused on a 6.5×10⁻³ cm² area of the devices through a lens and slit. The excitation wavelength was 337 nm, pulse width was 3.5 ns, and repetition rate was 20 Hz. The excitation light was incident upon the devices at around 20° with respect to the normal to the device plane. The emitted light was collected normal to the device surface with an optical fiber connected to a multichannel spectrometer (PMA-50, Hamamatsu Photonics) and placed 3 cm away from the device. Excitation intensities were controlled using a set of neutral density filters (motorized filter wheel FW102C). For the CW operation, a CW laser diode (NICHIYA, NDV7375E, maximum power of 1400 mW) was used to generate excitation light with an excitation wavelength of 405 nm. In these measurements, pulses were delivered using an acousto-optic modulator (AOM, Gooch & Housego) which was triggered with a pulse generator (WF 1974, NF Co.). The excitation light was focused on a 8.76×10⁻⁶ cm² area of the devices through a lens and slit, and the emitted light was collected using a multichannel spectrometer (PMA-50, Hamamatsu Photonics) or streak scope (C7700, Hamamatsu Photonics) with a time resolution of 100 μs that was connected with a digital camera (C9300, Hamamatsu Photonics). The emission intensity was recorded using a photomultiplier tube (PMT) (C9525-02, Hamamatsu Photonics). Both the PMT response and the driving square wave signal were monitored on a multi-channel oscilloscope (Agilent Technologies, MS06104A). The same irradiation and detection angles were used for this measurement as described earlier. The size of the excitation area was carefully checked by using a beam profiler (WimCamD-LCM, DataRay). All the measurements were performed in nitrogen atmosphere to prevent any degradation resulting from moisture and oxygen.

All three chromophores SFCz, BSFCz and BSTFCz showed lasing profiles with both second-order and mixed-order grating structures. The lasing thresholds of these spin-coated (6 wt % in TCTA) DFB lasers are summarized in Table 4.

TABLE 4
Lasing thresholds obtained from spin-coated mixed-order and
second-order DFB lasers based on blend thin films
(6wt % in TCTA) of either SFCz, BSFCz or BSTFCz.

| | Lasing Threshold | Lasing Threshold |
|---|---|---|
| | (μJ cm⁻²) | (μJ cm⁻²) |
| Chromophore | (mixed-order) | (second-order) |
| SFCz | 1.2 ± 0.2 | 1.2 ± 0.2 |
| BSFCz | 0.8 ± 0.2 | 0.9 ± 0.2 |
| BSTFCz | 0.5 ± 0.1 | 0.6 ± 0.1 |

It was found that for all three chromophores, slightly lower lasing threshold values were obtained with mixed-order grating structure, compared to second-order grating structure (Table 4). This indicates that mixed-order DFB works well compared to second-order DFB structure in order to reduce the lasing threshold. In particular, the lasing threshold (0.587 ρJ cm⁻²) obtained from mixed-order DFB structure of BSTFCz blend film is lower than that (0.646 ρJ cm⁻²) of second-order DFB structure, as well as its ASE threshold (0.7 ρJ cm⁻²). The FWHMs of the lasing obtained with mixed-order DFB structure and second-order grating structure are 0.53 and 0.47 nm, respectively. The lowest lasing threshold (0.587 ρJ cm⁻²) obtained in BSTFCz is consistent with the fact that among the three new dyes, this chromophore has the highest PLQY, the highest kᵣ, and the lowest τ values in blend film.

Further clarification of laser action was investigated by polarisation as well as by examining beam divergence of the laser output at below and above lasing threshold of the DFB device with BSTFCz blend films using near-field and far-field interference spectra. Near-field and far-field images and their spectra obtained below, near, and above threshold values clearly confirm that the surface emission lasing profile of BSTFCz are actual lasing.

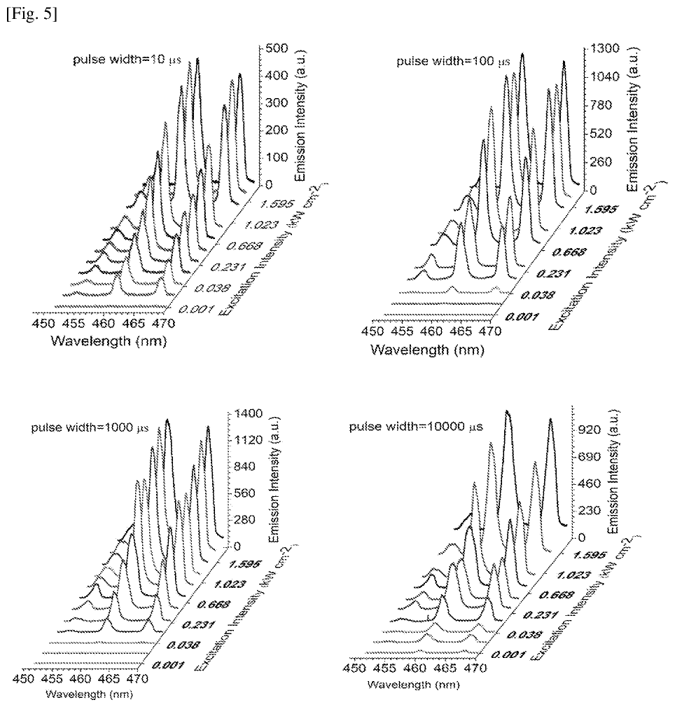

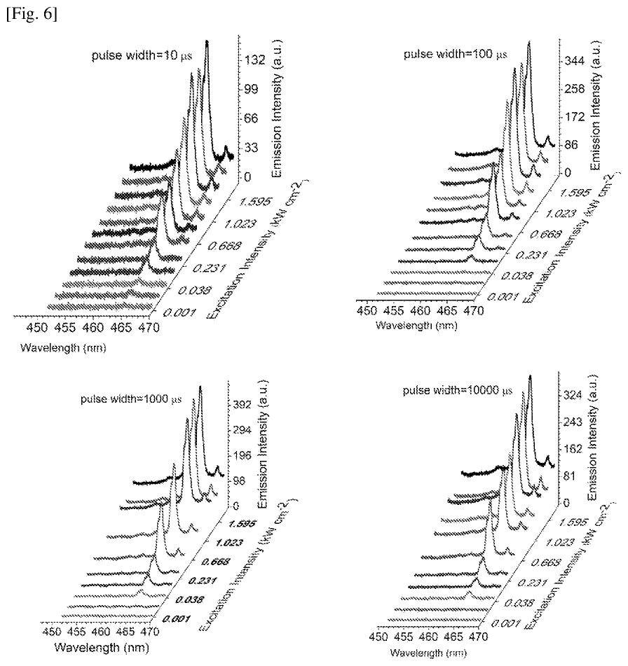

As BSFCz showed no overlap between triplet absorption in gain region (FIG. 3), we focused on BSFCz for further investigation in CW operation. The fabricated DFB devices using blend films of BSFCz were investigated for laser characteristics in the long pulse regime using inorganic laser diode which operates at 405 nm with maximum power of 1400 mW. The pulsed width was varied from 10 Is to 10 ms. The pumping intensity was varied from 0 to 2.7 kW cm⁻². This encapsulated DFB device worked properly giving rise laser action under long pulses of 10 μs to 10 ms which can be further extended to lengthier pulses. The temporal profile clearly shows that there is insignificant quenching due to singlet excited-states by singlet-triplet annihilation as the intensity of the PL stays the same after 10 μs irradiation. FIGS. 5 and 6 show the output intensity and emission spectra of BSFCz blend films with mixed-order grating structure and second-order grating structure respectively, as a function of pump intensity. Lasing threshold was calculated considering the abrupt change in the slope of Emission Intensity versus Excitation Intensity graph. The calculated threshold value appeared around 0.203 kW cm⁻² for DFB device with mixed-order structure and 0.235 kW cm⁻² for DFB device with second-order structure in the 10 μs pulse width operation (Table 5). Low-threshold surface-emitting organic distributed feedback lasers operating in the CW regime under long pulse photoexcitation of up to 10 ms was successfully demonstrated.

TABLE 5
Lasing threshold in varies DFB devices as a function
of pulse width in CW operation.

| | Lasing Threshold | Lasing Threshold |
|---|---|---|
| | (kW cm⁻²) | (kW cm⁻²) |
| Pulse width (μs ) | (mixed-order) | (second-order) |
| 10 μs | 0.203 | 0.235 |
| 100 μs | 0.210 | 0.286 |
| 1000 μs | 0.216 | 0.291 |
| 10000 μs | 0.224 | 0.300 |

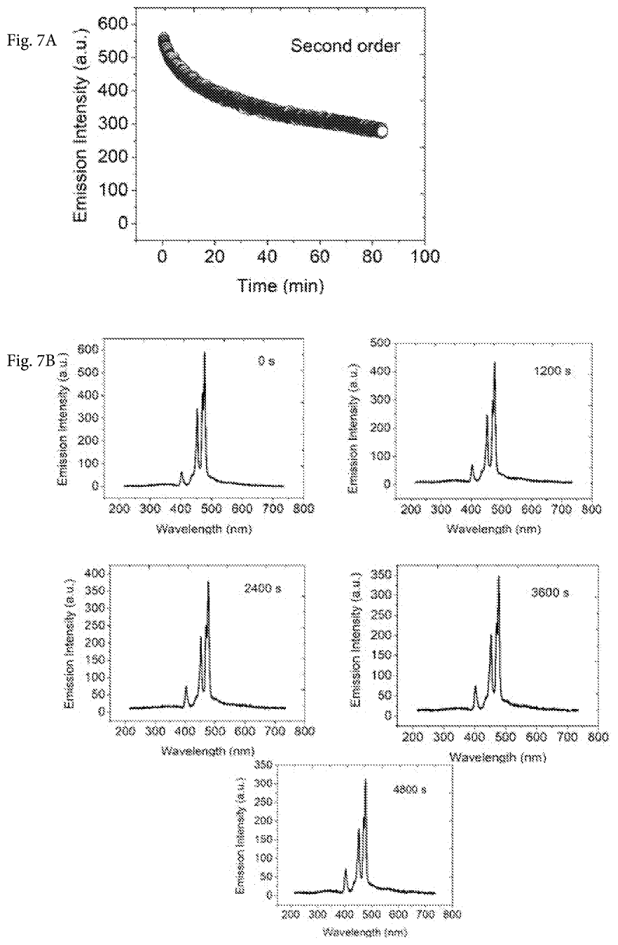

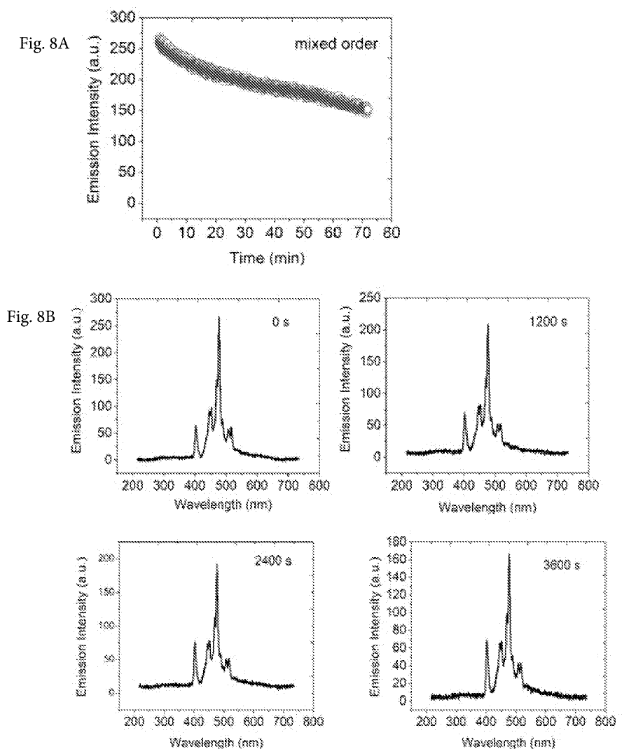

Moreover, when the stability of the BSFCz was checked with CW regime, the BSFCz molecule showed promising results in the time frame of more than 60 min with both second-order and mixed-order DFB structures. As shown in FIGS. 7 and 8, the laser emission stayed for long hours while decreasing its intensity. Also there were no significant broadening observed of the emitted laser peak in the long time exposure. BSFCz chromophore still has a window for future studies to obtain more lower threshold with high stability. Further, optimization of the resonator geometry and laser structure should lead to lower lasing thresholds and represent an important future direction for the development of a CW organic laser technology and for the realization of an electrically-pumped organic laser diode.

### INDUSTRIAL APPLICABILITY

A new series of high-performing solution-processable organic semiconductor dyes, SFCz, BSFCz and BSTFCz were successfully developed and synthesised. It was found that the elongated π-conjugation in the structure significantly enhances their molar extinction coefficients, radiative decay rates, as well as shortens their excited-state lifetimes. These desirable features have contributed to excellent ASE thresholds of the materials (ranging from 0.7 to 2.1 ρJ cm⁻²) in spin-coated blend films in a common TCTA host. The low ASE threshold values are comparable to current state-of-the-art organic laser dyes processed by vacuum deposit. Transient absorption spectroscopy measurements showed that among these three chromophores, BSFCz has minimal triplet absorption at its ASE wavelength. Distributed feedback lasers based on these three dyes were successfully fabricated on both second-order and mixed-order grating structures to give low lasing thresholds ranging from 0.6 to 1.2 ρJ cm⁻². The lasing profiles of these DFB lasers were confirmed by polarisation, as well as near-field and far-field interference effects. Finally, we successfully demonstrated solution-processed DFB grating lasers based on BSFCz with efficient lasing under long photoexcitation pulse up to 10 ms and with high stability. This invention thus has a high industrial applicability.

1. A compound represented by the following formula (1): 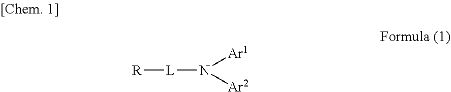
wherein:
Ar¹ and Ar² each independently represent a substituted or unsubstituted aryl group, and Ar¹ and Ar² may be bonded to each other;
L represents a divalent group having at least one phenyl rings wherein the divalent group consists of at least one group represented by the formula (2) below, optionally at least one group represented by the formula (3) below, and optionally at least one group represented by the formula (4) below: 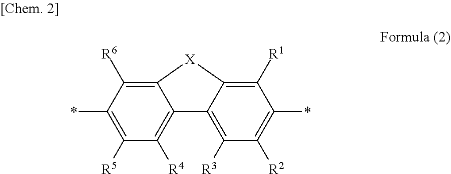
wherein X represents >C(R⁷)(R⁸), —O—, —S— or >N(R⁹); R¹ to R⁹ each independently represent a hydrogen atom or a substituent, R² and R³, and R⁴ and R⁵ may be taken together to form a ring, and each * represents a bonding site, 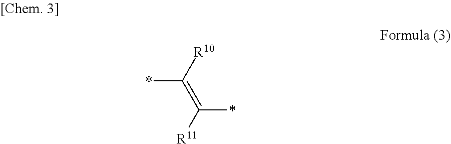
wherein R¹⁰ and R¹¹ each independently represent a hydrogen atom or a substituent, 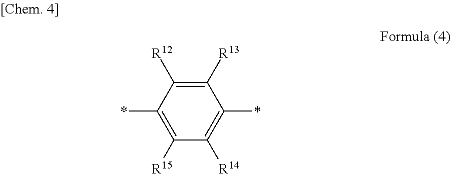
wherein R¹² to R¹⁵ each independently represent a hydrogen atom or a substituent, and R¹² and R¹³, and R¹⁴ and R¹⁵ may be taken together to form a ring,
R represents a hydrogen atom or a group represented by the following formula (5): 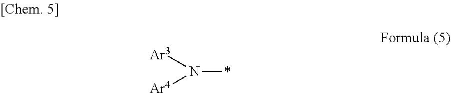
wherein Ar³ and Ar⁴ each independently represent a substituted or unsubstituted aryl group, and Ar³ and Ar⁴ may be bonded to each other; and
wherein at least one alkyl group having at least five carbon atoms which are bonded is represent in the formula (1).

2. The compound according to claim 1, wherein L is a divalent group consisting of at least one group represented by the formula (2), at least one group represented by the formula (3), and at least one group represented by the formula (4).

3. The compound according to claim 1, wherein L is a divalent group having a unit in which a group represented by the formula (2) and a group represented by the formula (3) are bonded.

4. The compound according to claim 1, wherein L is a divalent group having a unit in which a group represented by the formula (3) and a group represented by the formula (4) are bonded.

5. The compound according to claim 1, wherein L is a divalent group having a unit in which a group represented by the formula (2), a group represented by the formula (3) and at least one group represented by the formula (4) are bonded in this order.

6. The compound according to claim 1, having at least two alkyl groups having at least five carbon atoms which are bonded.

7. The compound according to claim 1, wherein L is a divalent group having at least one alkyl group having at least five carbon atoms which are bonded.

8. The compound according to claim 1, wherein X is >C(R⁷)(R⁸) or >N(R⁹) and R⁷ to R⁹ each independently represent an alkyl group having at least five carbon atoms which are bonded.

9. The compound according to claim 1, having two or more groups represented by the formula (2).

10. The compound according to claim 1, wherein —N(Ar¹)(Ar²) is a substituted or unsubstituted 9-carbazolyl group.

11. The compound according to claim 1, wherein R is a substituted or unsubstituted 9-carbazolyl group.

12. The compound according to claim 1, having a symmetrical structure.

13. The compound according to claim 1, having a structure represented by the following formula (6): 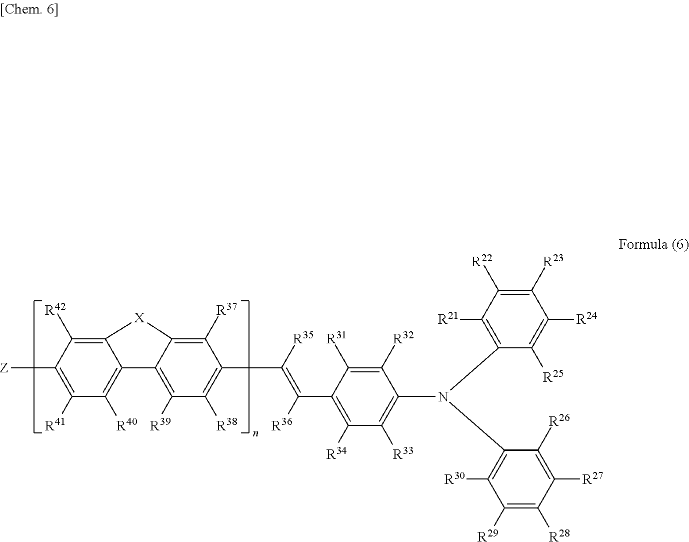
wherein X represents >C(R⁷)(R⁸), —O—, —S— or >N(R⁹), R⁷ to R⁹, R²¹ to R⁴² and Z each independently represent a hydrogen atom or a substituent, R²¹ and R²², R²² and R²³, R²³ and R²⁴, R²⁴ and R²⁵, R²⁵ and R²⁶, R²⁶ and R²⁷, R²⁷ and R²⁸, R²⁸ and R²⁹, R²⁹ and R³⁰, R³¹ and R³², R³³ and R³⁴, R³⁸ and R³⁹, and R⁴⁰ and R⁴¹ may be taken together to form a ring, and n is an integer of 1 to 10.

14. The compound according to claim 13, wherein Z is represented by the following formula (7): 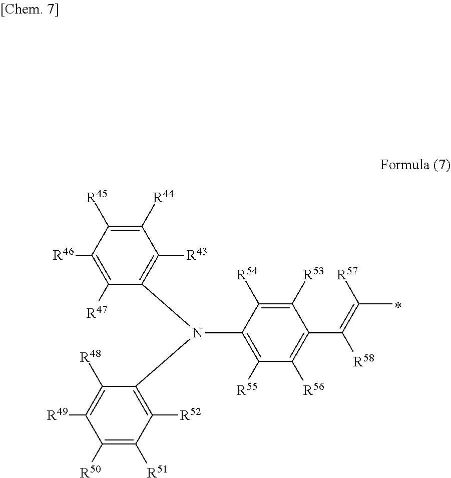
wherein R⁴³ to R⁵⁸ each independently represent a hydrogen atom or a substituent, R⁴³ and R⁴⁴, R⁴⁴ and R⁴⁵, R⁴⁵ and R⁴⁶, R⁴⁶ and R⁴⁷, R⁴⁷ and R⁴⁸, R⁴⁸ and R⁴⁹, R⁴⁹ and R⁵⁰, R⁵⁰ and R⁵¹, R⁵¹ and R⁵², R⁵³ and R⁵⁴, and R⁵⁵ and R⁵⁶ may be taken together to form a ring, and * represents a bonding site.

15. A method of manufacturing an organic semiconductor laser, the improvement comprising an emitter having the compound of claim 1.

16. An organic semiconductor laser comprising the compound of claim 1 as an emitter.

17. The organic semiconductor laser according to claim 16, having an optical resonator structure composed of a second-order Bragg scattering region.

18. The organic semiconductor laser according to claim 16, having an optical resonator structure composed of a mixed-order Bragg scattering region.

19. A method for producing an organic semiconductor laser comprising forming a layer having the compound of claim 1 by a solution process.
